(19) United States
(12) Patent Application Publication
(10) Pub. No.: US 2018/0280190 A1
(43) Pub. Date: Oct. 4, 2018

(54) TRI-MODAL LOCALIZED HEATING GARMENT

(71) Applicants: Max Betkowski, Larkspur, CA (US); Dannick Alokele Liudzius, Simi Valley, CA (US)

(72) Inventors: Max Betkowski, Larkspur, CA (US); Dannick Alokele Liudzius, Simi Valley, CA (US)

(21) Appl. No.: 15/474,822

(22) Filed: Mar. 30, 2017

(51) Int. Cl. A61F 7/02; A41D 13/005; A41D 27/20; A41D 3/00; A61F 7/00

(52) U.S. Cl. CPC A61F 7/02; A41D 13/0051; A41D 27/205; A41D 3/00; A61F 7/007; A61F 2007/0244; A61F 2007/0228; A61F 2007/0234; A61F 2007/0096; A61F 2007/0073; A61F 2007/0088

(57) ABSTRACT

A garment that heats a portion of a human body during warm-up prior to athletic endeavor having a heater pad member with a size between the size of a common index card and an ordinary sheet of paper for localized penetrating heating of tissue and muscles. Heating is achieved by electrical heating of a heater pad member having a reflective metal foil on one side of the heater pad and thermally conductive PVF sheets on the opposite side. On the one hand, the metal foil reflects optical radiation toward a human body, while on the other hand, the PVF sheets direct infrared radiation into the human body. Control electronics, a battery and the heater pad are contained in separate pockets of the garment that are electrically connected together.

### TECHNICAL FIELD

The invention relates to body heating and, in particular, to body heating for exercise warm-up and cool down.

### BACKGROUND OF THE INVENTION

In many sports, the body core or limbs attain an activity temperature well above normal body temperature. At the same time, skin pores open to allow more perspiration. It is desirable that the activity temperature be reached gradually to avoid thermal strain on muscles and nerves. To reach the activity temperature, athletes usually perform warm-up exercises, gradually increasing body temperature. Similarly, when sports activity ceases, the body returns to a normal temperature but, to avoid thermal strain on muscles and nerves, as well as to allow gradual closing of skin pores, athletes frequency wear coats or blankets to allow for gradual cool down.

Body heating and cooling devices of various types are known in the prior art. U.S. Pat. Nos. 8,445,819 and 7,115,844 to Ferguson describe a therapeutic heater element for topical application with electrical heating and programmable thermal regulation. As seen in the '819 patent, an adhesive retaining layer is laminated to the back surface of a fabric heater element, which in turn is flush with a wound contact layer. The flexible electric circuit that forms the heater may be a metalized fabric that has been photochemically etched. There is direct skin contact with a heated porous fabric.

U.S. Pat. No. 5,891,189 to Payne, Jr. describes a heating band or belt providing both heat and pressure to sore muscles. The band is an elongate elastomeric member wrapping about the body part to be heated. An electric heating coil member is embedded within the belt. Heat output is controlled by means of a temperature-controlled switch.

U.S. Patent Publication 2016/0213509 of Petitt describes a thermal wrap that further combines electrical nerve stimulation. Thermal media are sandwiched between plastic film and fabric.

U.S. Pat. No. 7,458,106 to Yue describes a glove having a heating element disposed inside of a fabric sleeve. The heating element is apparently less rigid or prone to break and so is both more comfortable for a user and more durable.

U.S. Pat. No. 6,915,641 to Harvie combines the features of both heating and cooling in a system in the form of a vest and belt worn by a user. Although it is electrically operated, the heat (or cooling) is conducted by circulating fluids (liquid or air) in channels through the vest.

U.S. Patent Publication 2014/0277220 of Brennan et al is likewise in the form of a garment worn by a user. It provides selective control over the areas of a patient's body to which heat can be applied. The whole system appears to be battery operated.

U.S. Patent Publication 2015/0290028 of Isserow et al. describes a battery-powered heat applicator for injury sites of a user. It is a multilayer structure with a heat source sandwiched between a power source and a heat-conductive layer and heat application surface.

While all of the heating garments, bands and pads of the prior art are useful, none are specifically designed or adapted to athletic performance pre-activity and post-activity body intermediate level heating. In order to assure that muscles, nerves and skin pores attain an initial desired level of pre-heating, a regulated heating device is needed that is specific for athletic performance. An object of the invention was to devise such a regulated heating device.

### SUMMARY

The above object has been met with a heated garment to be worn by an athlete at warm-up time or cool down time. The garment is a heated wrap that has a length that envelops a human body portion, such as a limb or the human torso, by means of an elastomeric band or tight jacket, allowing the wrap to firmly contact a human body portion, such as an arm or leg or body core, in order to communicate maximum heat, not only by contact but by radiation. The wrap has a heater pad pocket for supporting a heater pad that has a length that is a fraction of the band length, always less than half of the length. To amplify heating, the heater pad has a reflective foil member on a side of the pad away from the human body portion and laminated PVF sheets on the opposite side facing the human body portion for infrared heating of adjacent human tissue and muscles. On the one hand, the reflective foil member transmits optical radiation towards a body portion, while, on the other hand, the laminated PVF sheets transmit infrared radiation from the heater pad a short distance into the body portion, penetrating up to several inches into the body. In this manner heat is transmitted to a body portion in a tri-modal manner, namely direct contact, optical radiation and infrared radiation.

Besides the heater pad pocket, the wrap has pockets containing power and control elements. A power pad pocket holds a removable flat battery that communicates power to the heater pad. A controller pad pocket has electronics, including a thermistor and a switch connected between the battery and the heater pad in a normally ON configuration, except that the switch may be turned OFF manually. The thermistor or similar device regulates temperature to achieve a predetermined and calibrated maximum temperature. The controller pad pocket may also support an external, user manipulated selector that allows manual selection of one temperature of a series of temperatures in a range of maximum temperatures between 110 degrees F. and 135 degrees F. The selector, connected to auxiliary resistors, governs the amount of current sourced by the thermistor for achieving the desired maximum temperature.

A garment such as a tightly fitting jacket can support more than one heater pad, with each heater pad associated with a controller pad pocket and a power supply pocket.

### BRIEF DESCRIPTION OF THE DRAWINGS

FIG. 1 is a front plan view of a first embodiment of a garment of the invention.

FIG. 2 is a back plan view of the garment of FIG. 1.

FIG. 3 is a sectional plan and electrical diagram of a portion of the garment of FIG. 1.

FIG. 4 is an exploded top view of a heater pad for use with the garment of FIG. 1.

FIG. 5 is a front plan view of a second embodiment of a garment of the invention.

### DETAILED DESCRIPTION

With reference to FIG. 1 a garment 11 is seen to be a wrap member 13 resembling a belt closely fitting around a human torso and a width of approximately 8 inches. It is not necessary for the wrap member to extend about a human torso. Smaller versions of the invention could wrap around a limb, namely an area or leg. A wrap member 13 has an outer surface 15 facing away from a human torso. Within the wrap member are three pockets. A first pocket is a heater pad pocket 17 holding a heater pad. Dashed lines are shown for the pockets because they are not visible from the front of the belt. A second pocket is the controller pocket 19, housing electronic components, which is closely adjacent to the heater pad pocket 15 being separated by about an inch or less. A user temperature selection switch 44, discussed below, is associated with the controller pocket 19. A third pocket is a power pack pocket 21 which is adjacent to the controller pocket. An ON-OFF toggle switch 22, discussed below, is associated with the power pack pocket. The pockets have wires extending between them described below with reference to FIG. 3.

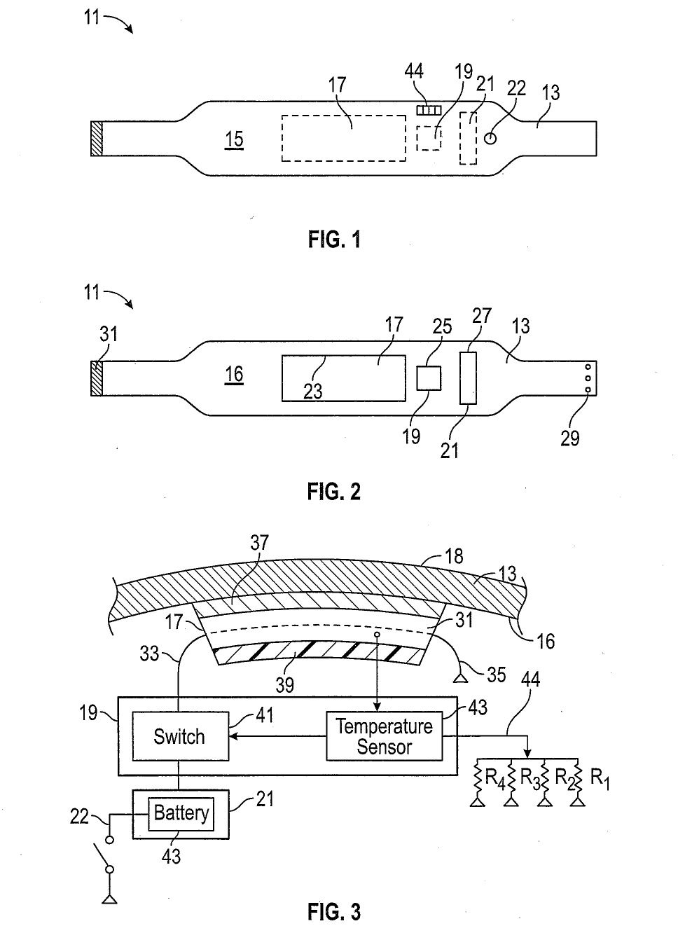

With reference to FIG. 2, the back side of garment 11 shows the outline of pockets 17, 19, and 21 which are within the inner surface 16 of wrap member 13 holding various components of the garment. The heater pad pocket 17 supports a heater pad member that is sandwiched between a metal foil and a plurality of lamented polyvinyl fluoride (PVF) sheets. The foil member in the PVF sheets amplify heat generated in the heater pad member by energy conversion to infrared radiation, directing infrared radiative heat into a human body portion that is adjacent to the heater pad pocket. The pocket has an opening along edge 23 so that the heater pad sandwich can be removed and replaced with a different heater pad sandwich having a different temperature characteristic. A desired temperature for athletic warm ups may be a selected temperature in the range of 110 degrees F.-135 degrees F., for example selected among 110 degrees, 120, 130 and 135 degrees with a user manipulated switch on the pocket. Electrical connectors between controller electronics in an adjacent controller pocket 19 allow for electrical communication between pockets. The controller pocket 19 is closed along an edge 25 to prevent tampering and to keep control electronics dry, thereby avoiding shorting. A similar construction exists for power pack pocket 21 having a sealed edge 27 protecting a battery that communicates power to the controller pocket 19 which, in turn, communicates power to the heater pad member. The power pack pocket 21 is located a close distance to the control pocket. Each of the controller pocket and power pack pocket has an external, user selectable switch.

Snaps 29 allow wrapping the wrap member 13 around the human torso in a tight-fitting manner. It is intended that the wrap member 13 be made of an elastomeric fabric such as spandex or Lycra, a registered trademark of Invista, both elastic polyurethane fibers, so that the wrap member will be tight fitting when snaps 29 are joined to snap fasteners 31 in a belt manner.

With reference to FIG. 3 the wrap member 13 has a inner surface 16 supporting heater pad pocket 17. Inner surface 16 faces a human torso, as in FIG. 2, while outer surface 15 faces outwardly as in FIG. 1. Heater pad pocket 17 contains a sandwich of three members. The principal member is a heater pad member 31 which has a heater wire, such as a carbon fiber filament or a nichrome wire, with a first end 33 connected to controller pocket 19. A second end 35 of the heater wire is grounded, making a return current path, not shown. Carbon fiber 12-volt heater pads used as automotive seat heaters, sold by Mito Corporation, can be cut to a desired size. Carbon fiber heater pads are described in U.S. Published Appl. No. 2008/0156786 and in U.S. Pat. No. 6,483,087. The carbon fiber heater pad of Appl. No. 2008/0156786 is said to emit radiation in the far infrared which is desirable in the present invention.

On a first major side of the heater pad 31 is a shiny, reflective foil member 37. The foil member may aluminum foil, or preferably a silver foil, having a thickness of only a few thousandth of a inch. The shiny side of the reflective foil member 37 faces the heater pad member 31 for directing optical radiation away from wrap member 13 and towards the human body enclosed by the wrap member.

On the opposite side of the heater pad member is a plurality of laminated PVF (polyvinyl fluoride) sheets 39 whose function is to convert thermally transmitted heat to far infrared radiation which is directed into a human body that the wrap member 13 surrounds. Both the metal foil member 37 and the PVF sheets 39 have approximately the same areawise dimensions of the heater pad member 31 such that the metal foil member, the heater pad member, and the PVF sheets form a sandwich within heater pad pocket 17 that may be laminated together or left as separate members. Typical areawise dimensions for the members is approximately 4 inches in height (width) and 8 inches in length. Regarding area dimensions of the heater pad, preferred width should not exceed 8 inches and preferred length should not exceed 10 inches, i.e. less than the size of a sheet of paper and larger than the size of a common index card. While the wrap member 13 has a thickness of approximately 3 millimeters, the heater pad member has a thickness of less than 2 millimeters. A combination, the wrap member and the heater pad pocket 17 have a combined thickness of approximately 5 millimeters or less as a preferred dimension. This preferred dimension is not essential and other thicknesses could be selected.

Controller pocket 19 contains a normally ON switch 41 receiving wire 33 from the heater pad member 31. The switch 41 supplies electrical current to the heater pad member 31 from a low voltage battery 43 which is in the closed power pack pocket 21. The battery 43 is preferably a rechargeable battery of the type used in cameras or smart phones. An external user toggle switch 22 can manually turn the battery 43 to an OFF state, although the battery is normally kept ON when the garment is in use.

Within the controller pocket 19 a temperature sensor 43, such as a thermistor measures the temperature in the heater pad member 31 and regulates current into the heater pad to maintain a selected temperature. Other temperature devices, such as thermostats or thermocouples may be substituted with appropriate ancillary support circuits. The heater pad member is built to achieve a maximum temperature, such as in the range of 110 degrees F. to 135 degrees F. The thermistor regulates current into the wire 33 which feeds the serpentine heater wire or carbon fiber wire in the heater member 31. The temperature sensor 43 may be built into the switch 41 and is not necessarily a separate component. In either situation, these electrical members are in the controller pocket 19.

The thermistor controls current to the heater pad. A logic chip is associated with a user switch that adjusts resistance into the heater pad to achieve a selected temperature. A user manipulated external switch 44, allows selection of an auxiliary resistor R1, R2, R3 or R4 that correspond to one of a selected maximum temperature, for example, 110, 120, 130 or 135 degrees F. set by thermistor temperature sensor 43.

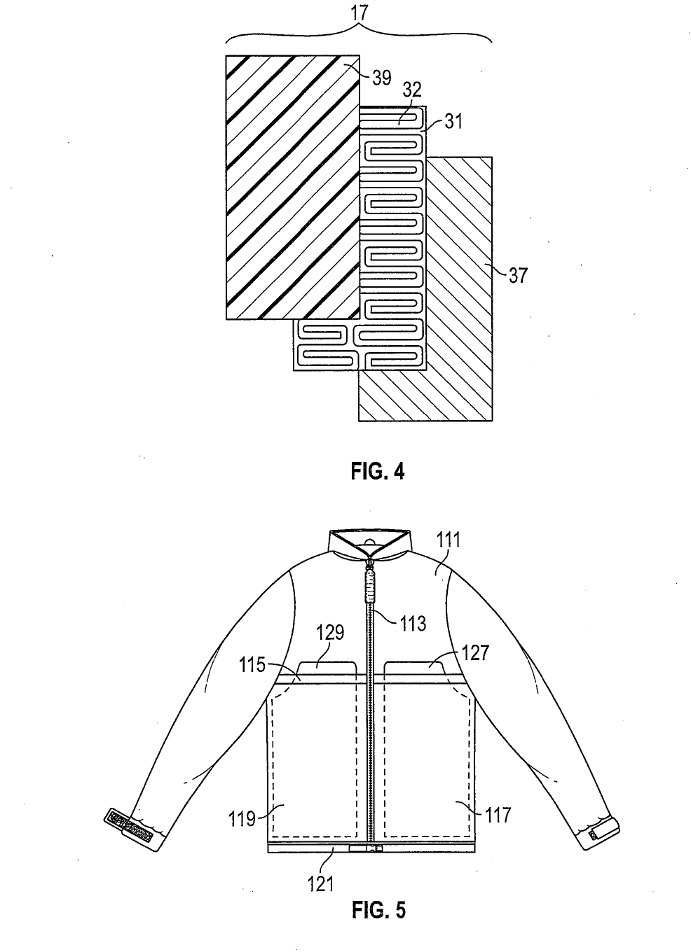

The contents of heater pad pocket 17 are shown in FIG. 4 and include the shiny reflective metal foil member 37, the heater pad member 31 carrying a serpentine pattern of wires 32, sandwiched by the polyvinyl sheets 39 which could be a plurality of thin sheets joined together, with a total thickness of a fraction of one millimeter. The foil member 37, the heater pad member 31 and the polyvinyl sheets 39 may be laminated together.

FIG. 5 shows an alternative garment 111, namely a jacket, that wraps around a human torso and has a slide fastener 113 in front, as well as circumferential belts 115 and 117. Between the belts, a pair heater pad pockets 117 and 119 may be disposed, with each heater pad pocket associated with a corresponding power pack pocket and controller pocket both of which are contained in regions 127 and 129, respectively, adjacent to the heater pad pockets. The heater pad pockets, the power pack pockets and the controller pockets all work as previously described. The jacket embodiment of FIG. 5 can be used not only for athletic warm-ups, but by motorcycle riders and participants in cold weather sports. The garment of the present invention is not restricted to use the torso, or on any particular limb, but may be used on a shoulder, neck or foot, or wherever localized penetrating heat is needed.

What is claimed is:

1. A garment for localized heating of a human body member comprising:
an elastomeric wrap member having opposed outer and inner major surfaces, the wrap member having a length that envelops a human body portion with the outer major surface facing away from the body portion;
a heater pad member supported within a heater pad pocket in the wrap member, having an electrically insulative and thermally conductive fabric confining a heater element with first and second ends, the heater pad member having a lengthwise dimension that is less than half the length of the wrap member;
a shiny, reflective metal foil member within the heater pad pocket of the wrap member with a first foil surface facing heater pad member and a second foil surface facing the inner major surface of the wrap member, with areawise foil member dimensions generally corresponding to areawise dimensions of the heater pad member;
a plurality of laminated PVF sheets forming a infrared radiation transmitting member generally corresponding in areawise dimensions to the heater pad member, the PVF sheets having a combined thickness of less than one millimeter, and having opposed major surfaces with one major surface facing the heater pad, and one major surface arranged for facing a human body member, the foil member and PVF sheets sandwiching the heater pad member therebetween, all within the heater pad pocket;
at least one closed power pack pocket in the wrap member containing a removable flat battery, the power pack pocket laterally near the flexible heater pad pocket, the battery having a battery electrode electrically communicating power to a first end of the heater element of the flexible heater pad member; and
a closed controller pocket with control electronics electrically connected between the power communicating battery electrode and the first end of the heater element, and a temperature sensor member associated with the controller pocket producing electrical signals governing temperature, the sensor having a temperature set point indicative of a predetermined maximum temperature so that the wrap member maintains a body member near the predetermined maximum temperature;
whereby the reflective foil directs optical radiation from the heater pad member towards a body member and the PVF sheets direct infrared radiation towards the body member, while the heater pad thermally conducts heat towards the body member.

2. The garment of claim 1 wherein the wrap member has a belt shape.

3. The garment of claim 1 wherein the wrap member has a jacket shape.

4. The garment of claim 3 having two heater pad members.

5. The garment of claim 1 wherein the temperature sensor member is a thermistor.

6. The garment of claim 1 wherein the temperature sensor is connected to an auxiliary resistor array for achieving a user-selected maximum temperature.

7. The garment of claim 1 wherein the heater element is a carbon fiber heater.

8. The garment of claim 1 wherein the heater element is a serpentine nichrome wire.

9. The garment of claim 1 where the wrap member is made of elastic polyurethane fibers.

10. The garment of claim 1 wherein the heater pad member is less than the size of an ordinary paper sheet and greater than the size of a common index card.

11. A garment for localized heating of a human body member comprising:
an elastomeric wrap member having a thickness not exceeding 4 mm. and having opposed outer and inner major surfaces, the wrap member having a length that envelops a human body portion with the outer major surface facing away from the body portion;
a heater pad member supported within a heater pad pocket in the wrap member, with a thickness of less than 2 mm and having a non-woven electrically insulative and thermally conductive fabric confining a heater element with first and second ends, the heater pad member having a lengthwise dimension that is less than half the length of the wrap member;
a shiny, reflective metal foil member within the heater pad pocket of the wrap member with a first foil surface facing heater pad member and a second foil surface facing the inner major surface of the wrap member, with areawise foil member dimensions generally corresponding to areawise dimensions of the heater pad member;
a plurality of laminated PVF sheets forming a infrared radiation transmitting member generally corresponding in areawise dimensions to the heater pad member, the PVF sheets having a combined thickness of less than one millimeter, and having opposed major surfaces with one major surface facing the heater pad, and one major surface arranged for facing a human body member, the foil member and PVF sheets sandwiching the heater pad member therebetween, all within the heater pad pocket;
at least one closed power pack pocket in the wrap member containing a removable flat battery, the power pack pocket laterally near the flexible heater pad pocket, the battery having a battery electrode electrically communicating power to a first end of the heater element of the flexible heater pad member; and
a closed controller pocket with control electronics electrically connected between the power communicating battery electrode and the first end of the heater element, and a temperature sensor member associated with the controller pocket producing electrical signals governing temperature, the sensor having a temperature set point indicative of a predetermined maximum temperature so that the wrap member maintains a body member near the predetermined maximum temperature;
whereby the reflective foil directs optical radiation from the heater pad member towards a body member and the PVF sheets direct infrared radiation towards the body member, while the heater pad thermally conducts heat towards the body member.

12. The garment of claim 11 wherein the wrap member has a belt shape.

13. The garment of claim 11 wherein the wrap member has a jacket shape.

14. The garment of claim 13 having two heater pad members.

15. The garment of claim 11 wherein the temperature sensor member is a thermistor.

16. The garment of claim 11 wherein the temperature sensor is connected to an auxillary resistor array for achieving a user-selected maximum temperature.

17. The garment of claim 11 wherein the heater element is a carbon fiber heater.

18. The garment of claim 11 wherein the heater element is a serpentine nichrome wire.

19. The garment of claim 11 where the wrap member is made of elastic polyurethane fibers.

20. The garment of claim 11 wherein the heater pad member is less than the size of an ordinary paper sheet and greater than the size of a common index card.
